(19) United States
(12) Patent Application Publication
(10) Pub. No.: US 2012/0172722 A1
(43) Pub. Date: Jul. 5, 2012

(54) ULTRASOUND APPARATUS AND GRAPHICAL INTERFACE FOR PROCEDURAL ASSISTANCE

(76) Inventors: Timothy Mark Chinowsky, Seattle, WA (US); Joshua Mikhael Kornfeld, Seattle, WA (US); Jeffrey William Ladwig, Seattle, WA (US); Austin Rand Porter, Seattle, WA (US)

(21) Appl. No.: 13/223,161

(22) Filed: Aug. 31, 2011

Related U.S. Application Data
(63) Continuation of application No. 12/986,143, filed on Jan. 6, 2011.
(60) Provisional application No. 61/293,004, filed on Jan. 7, 2010.

(51) Int. Cl. A61B 8/13; G06F 3/00; G06F 3/048; A61B 8/00; G09B 23/28

(52) U.S. Cl. 600/439; 600/443; 434/262; 715/781; 715/705

(57) ABSTRACT

A system includes an ultrasound transducer and a processing device coupled to the transducer and configured to generate to a display device a graphical user interface. The processing device is further configured to generate to a first region of the user interface an ultrasound image of a patient region of interest, generate to the first region at least one overlay image configured to indicate a specific feature associated with the region of interest, generate to a second region of the user interface a first set of selectable soft keys, the second region being exclusive of the first region, and in response to user selection of a first-set soft key, generate to a third region of the user interface a corresponding functional image.

### CROSS REFERENCES TO RELATED APPLICATIONS

This application is a continuation of U.S. patent application Ser. No. 12/986,143, filed Jan. 6, 2011, which in turn claims priority to U.S. Provisional Patent Application Ser. No. 61/293,004 filed Jan. 7, 2010. Each of these patent applications is incorporated by reference in its entirety as if fully set forth herein.

### BACKGROUND OF THE INVENTION

Medical personnel can be faced with patients who present arteries or veins that are difficult to access with a needle and any needle-cannula assembly due to the qualities of the overlaying skin and/or the size and configuration of a given artery or vein, and the techniques undertaken to access a given blood vessel. The vein or artery may be obscured due to overlying fatty tissues, or lack of sufficient blood flow may insufficiently till the lumen to make the blood vessel palpable, as may occur in the cases of blown veins compromised with a hematoma, veins that are otherwise structurally compromised as found in the elderly, users of intravenously administered drugs, and critically ill patients with very low blood pressure.

Such patients as these, as well as obese patients, prove difficult to cannulate under “blind” procedures. In many cases these patients have to endure multiple stabs with a needle, sometimes with penetration through the posterior wall of a vein before a successful placement of the needle is achieved and stable residence of the cannula or catheter within the blood vessel is achieved. Even allowing for an occasionally successful blind stick-and-insert catheter operation, the inserted catheter, if entered at too sharp an angle into a given blood vessel, may kink on insertion and thus hamper fluid delivery into or removal from the blood. vessel lumen.

Moreover, current ultrasound-image-guided blood-vessel-access procedures require two people: one person to hold the ultrasound probe to secure a guiding image, and another person to insert the needle/cannula. Accordingly, there is a need for solutions, for accessing blood vessels and other bodily structures, that do not require two people to perform, and that are more precise than are offered by current devices and procedures.

### SUMMARY OF THE INVENTION

In an embodiment, a system includes an ultrasound transducer and a processing device coupled to the transducer and configured to generate to a display device a graphical user interface. The processing device is further configured to generate to a first region of the user interface an ultrasound image of a patient region of interest, generate to the first region at least one overlay image configured to indicate a specific feature associated with the region of interest, generate to a second region of the user interface a first set of selectable soft keys, the second region being exclusive of the first region, and in response to user selection of a first-set soft key, generate to a third region of the user interface a corresponding functional image.

### BRIEF DESCRIPTION OF THE DRAWING

Preferred and alternative embodiments of the present invention are described in detail below with reference to the following figures:

FIG. 1 is a perspective view of a region-of-interest access handset in accordance with an embodiment of the invention;

FIG. 2 is a perspective view of the handset device of FIG. 1 equipped with a detachably attachable cartridge or cassette in accordance with an embodiment of the invention;

FIG. 3 is a perspective view of the handset device in accordance with an embodiment of the invention placed on a patient's arm;

FIG. 4 is a perspective view of an embodiment of an ultrasound system deployed from a movable cart;

FIG. 5 illustrates a touch screen monitor presenting a home screen of a user interface in accordance with an embodiment of the invention;

FIG. 6 illustrates the handset surveying for a peripheral vein to facilitate an IV procedure in accordance with an embodiment of the invention;

FIG. 7 schematically depicts an ultrasound image presented the context of a user interface in accordance with an embodiment of the invention;

FIGS. 8-9 illustrate instructional images in accordance with an embodiment of the invention;

FIG. 10 illustrates a screenshot of a user interface in accordance with an embodiment of the invention;

FIG. 11 illustrates the manner in which position indicators are calculated according to an embodiment of the invention; and

FIG. 12 illustrates a graphical user interface in accordance with an embodiment of the invention.

### DETAILED DESCRIPTION OF THE PREFERRED EMBODIMENT

Embodiments of the invention are operational with numerous general purpose or special purpose computing system environments or configurations. Examples of well known computing systems, environments, and/or configurations that may be suitable for use with the invention include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

Embodiments of the invention may be described in the general context of computer-executable instructions, such as program modules, being executed by a computer and/or by computer-readable media on which such instructions or modules can be stored. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

Embodiments of the invention may include or be implemented in a variety of computer readable media. Computer readable media can be any available media that can be accessed by a computer and includes both volatile and nonvolatile media, removable and non-removable media. By way of example, and not limitation, computer readable media may comprise computer storage media and communication media. Computer storage media include volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, versatile disks (DVD) or other optical disk storage, magnetic cassettes, magnetic tape, magnetic disk storage or other magnetic storage devices, or any other medium which can be used to store the desired information and which can accessed by computer. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

According to one or more embodiments, the combination of software or computer-executable instructions with a computer-readable medium results in the creation of a machine or apparatus. Similarly, the execution of software or computer-executable instructions by a processing device results in the creation of a machine or apparatus, which may be distinguishable from the processing device, itself, according to an embodiment.

Correspondingly, it is to be understood that a computer-readable medium is transformed by storing software or computer-executable instructions thereon. Likewise, a processing device is transformed in the course of executing software or computer-executable instructions. Additionally, it is to be understood that a first set of data input to a processing device during, or otherwise in association with, the execution of software or computer-executable instructions by the processing device is transformed into a second set of data as a consequence of such execution. This second data set may subsequently be stored, displayed, or otherwise communicated. Such transformation, alluded to in each of the above examples, may be a consequence of, or otherwise involve, the physical alteration of portions of a computer-readable medium. Such transformation, alluded to in each of the above examples, may also be a consequence of, or otherwise involve, the physical alteration of, for example, the states of registers and/or counters associated with a processing device during execution of software or computer-executable instructions by the processing device.

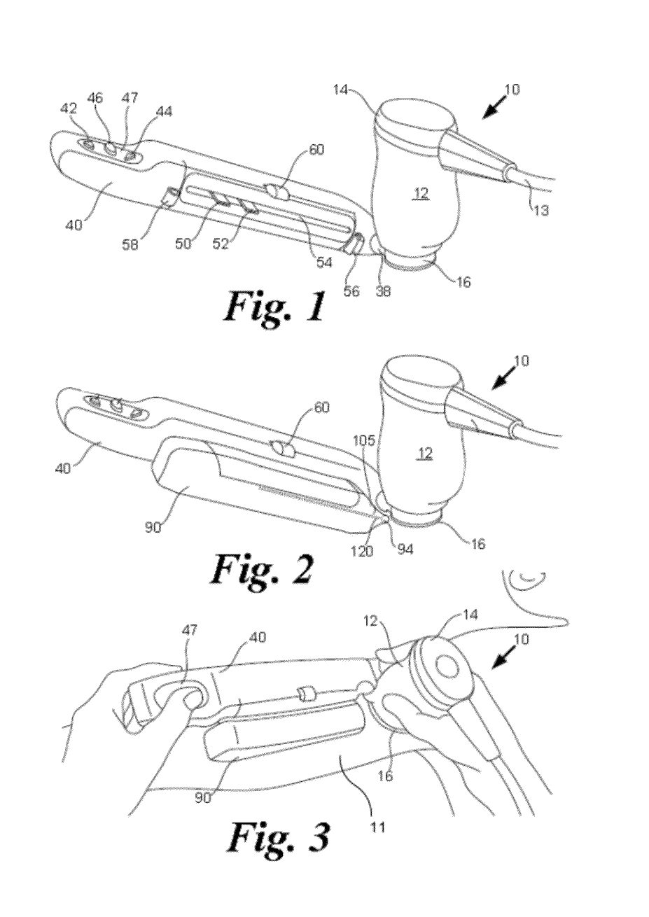

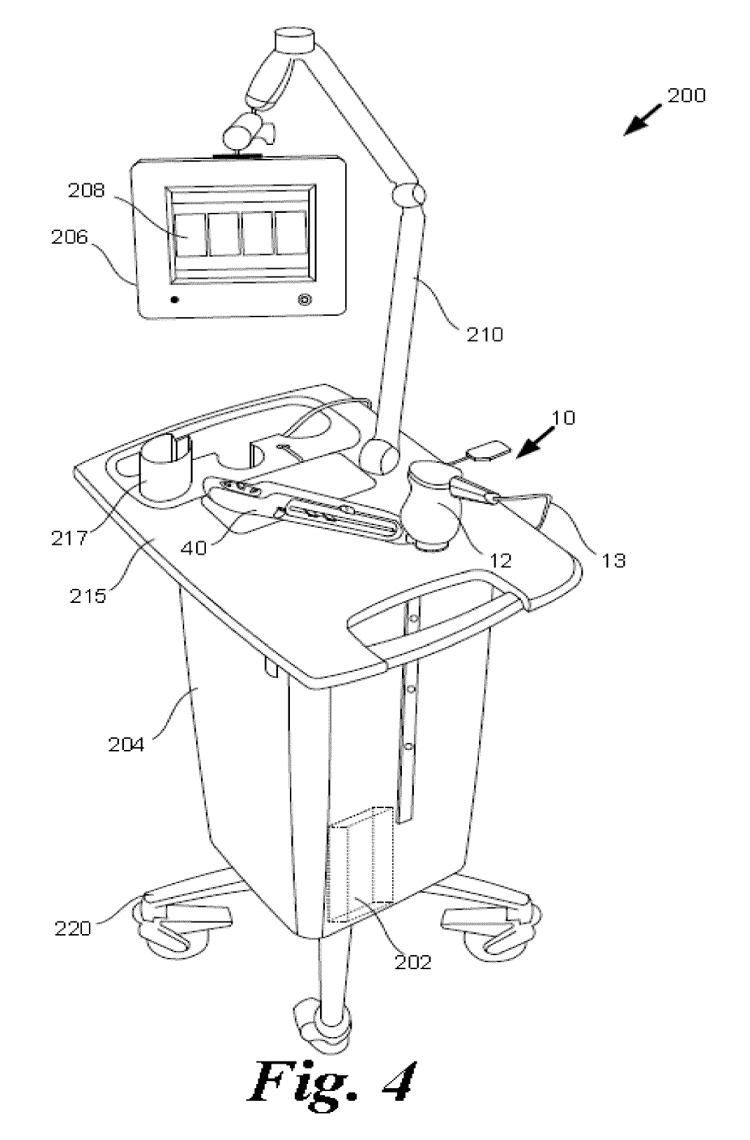

FIG. 1 is a perspective view of a region-of-interest access handset 10, according to an embodiment, that provides images of internal bodily structures utilizing, for example, B-mode based single scan planes and/or rotationally-configured scan plane arrays. The handset 10 includes an ultrasound transceiver housing 12 in communication with a central processing unit (FIG. 4) via power and data communication cable 13. The transceiver housing 12 includes a swiveling portion that allows the transceiver housing 12 to be grasped by righted-handed or left-handed users. Transceiver housing 12 includes a top 14 and transducer base 16. Attached in pivotable contact with the transducer base 16 is a friction hinge housing 38 that connects an injector arm 40 to the transceiver housing 12 via the transducer base 16.

The injector arm 40 is equipped with a controller 47 having a rearward-located pushbutton control 42, a forward-located pushbutton control 44, and a 4-way toggle control 46. In signal communication with the push and toggle buttons 42, 44, and 46 of controller 47 are motorized moveable platforms 50 and 52 that slidably transit along the length of a slot 54. Rearward control 42 retracts the moveable platform 50 away from the patient's region-of-interest independently of the position of the moveable platform 52. Forward control 44 moves the moveable platform 52 towards the patient's targeted region-of-interest independently of the position of the moveable platform 50. The 4-way toggle control 46 synchronously moves the moveable platforms 50 and 52 together toward the patient's region-of-interest if toggled towards the patient, and synchronously together away from the patient's region-of-interest if toggled away from the patient. Adjacent to the slot 54 are cassette holders 56 and 58. As shown in FIG. 1, the motorized platforms 50 and 52 occupy the distal third portion of the slot 54 away from the patient denoted as the “home” or “start” position within slot 54.

FIG. 2 is a perspective view of the handset device 10 of FIG. 1 equipped with a detachably attachable cartridge or cassette 90 to the slot 54 side of the injector arm 40 by engagement with cassette holders 56, 58 and moveable platforms 50, 52. Moveable platform 50 detachably engages with a slideable needle mount (not shown) and moveable platform 52 detachably engages with a slideable cannula mount (not shown) when the slideable mounts are positioned within the cassette 90 in the “home” or “start” position that is dimensionally accommodating or orientationally equivalent to the “home” and “start” positions of the motorized platforms 50 and 52 described with regard to FIG. 1 above.

As depicted in FIG. 2, cartridge 90 includes a needle guide 94 at the end near the support base 16. An aperture of the needle guide 94 serves to prevent significant sideways slippage of the needle 120 and/or overlapping cannula 140 as they pass through the needle guide. As shown in FIG. 2, the mounted needle 120 is depicted as a pair of dashed lines suspended internally within the cassette 90. Used cassettes 90 may be easily detached from injector arm 40 by pressing cartridge release button 60.

FIG. 3 is a perspective view of the handset 10 placed on a patient's arm 11. The handset 10 includes the cassette 90 attached to the slot 54 side of the injector arm 40 during a cannula placement operation into the patient's peripheral vasculature. In the illustrated embodiment, the transceiver housing 12 is pivoted for right-handed holding of the transducer base 16 against the patient's arm 11. The left hand of the user operates the tilting of the injector arm 40 about the friction hinge housing 38 and operation of the push and toggle buttons 42, 44, and 46 of controller 47 depicted in FIG. 1 above.

FIG. 4 is a top perspective view of an embodiment of an ultrasound system 200 deployed from a movable cart. The system 200 includes a monitor 206 equipped with a touch-sensitive screen 208. The monitor 206 is supported by an articulating arm 210 extending from a countertop 215 on which the handset 10 can be prepared for various region-of-interest access procedures. The power supply and communication cable 13 can conveniently access a computer having a central processing unit 202 disposed, for example, within cart support 204. Alternatively, or additionally, the central processing unit 202 may be integral to the monitor 206. The central processing unit 202 is configured to receive and process echoes of ultrasound signals to present images of insonified vasculatures. Included in the countertop 215 is a handset holder 217. The system 200 with handset 10, monitor 206, and central processing unit 202 may be conveniently rolled via wheeled extensions 220.

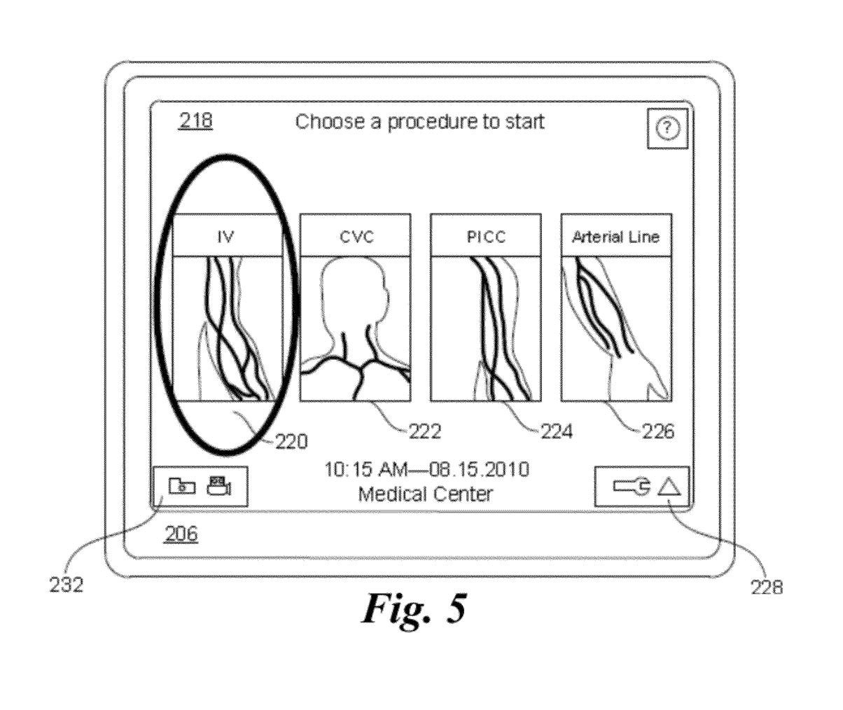

FIG. 5 illustrates the touch screen monitor 206 presenting a home screen 218 of a user interface, generated by unit 202, illustrating a panel of four blood-vessel-based access procedures characterized by different icons and acronyms. As stated previously, monitor 206 may be a touch screen enabling a user to interact with the user interface displayed thereon. Additionally, in an embodiment, the user may interact with any user interface displayed on monitor 206 using one or more controls associated with handset 10, discussed herein above. The panel of icons (soft keys) representing blood-vessel access procedures includes a peripheral intravenous IV procedure 220, a central venous catheter CVC procedure 222, a peripherally inserted central catheter PICC procedure 224, and an arterial tine procedure 226. In the case of a touch screen monitor 206, the IV procedure 220 icon is touched by the user, indicated by the oval, to bring up menu items to conduct this blood vessel access procedure. Also shown are touch sensitive tool icon 228 and data output icon 232. Selection of the tool icon 228, in an embodiment, may invoke display on the screen 218 of a configuration screen (not shown) allowing the user to customize conventional settings such as, for example, text-language selection and screen brightness. Selection of the data output icon 232, in an embodiment, may invoke a review screen (not shown) wherein images captured during previous procedures can be viewed.

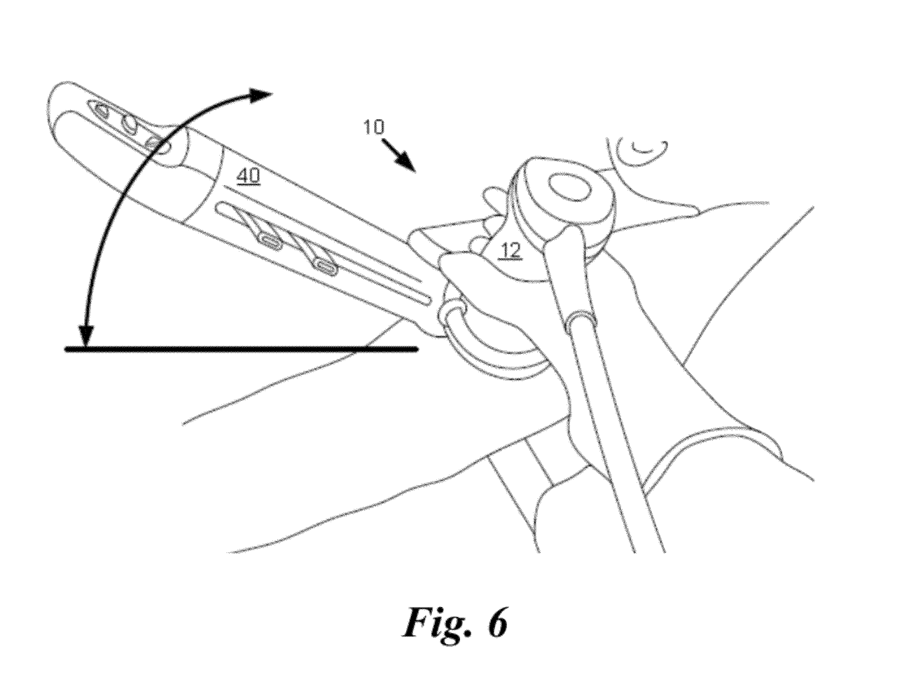

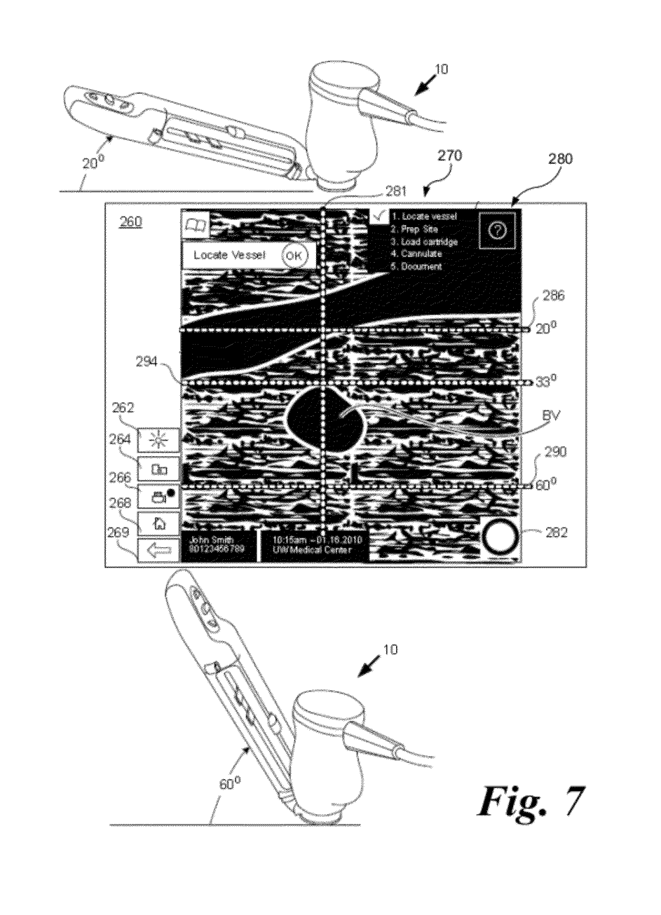

FIG. 6 illustrates the handset 10 surveying for a peripheral vein to facilitate the IV procedure selected from the home screen 218 depicted in FIG. 5. The injector arm 40 can pivot freely from shallow acute angles to steep acute angles in relation to the transceiver base 16 as denoted by the arrow in FIG. 6.

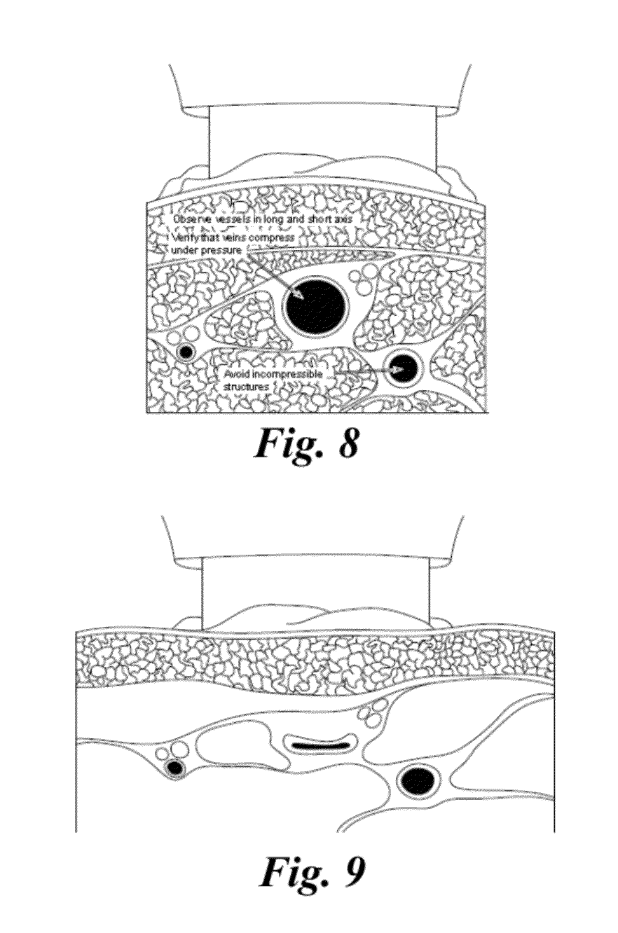

FIG. 7 schematically depicts an ultrasound image presented in the context of a user interface 260 on the monitor 206 while surveying for a blood vessel during a short-axis mode when a scan plane emanating from the handheld 10 intersects a blood vessel BV1 substantially at a perpendicular orientation. Interface 260 may include a brightness icon 262, a still-capture icon 264, a movie-capture icon 266, a home-return icon 268, and a return-to-prior-screen icon 269. In an embodiment, at key points (e.g., when the procedure step changes, before the transducer rotates, when the needle changes direction, etc.) throughout a given procedure, the processor 202 automatically takes screen shots of the ultrasound image. Consequently, at the end of a procedure, the user will always have a record of the procedure, even if they did not use the touch screen to request specific screen shots.

Upon selection of a procedure represented by the icons 220, 222, 224, 226 illustrated in FIG. 5, interface 260 displays a procedure-specific checklist 270 to assist the user in successfully completing each required task of such procedure. This checklist 270 may be displayed as an overlay relative to the displayed ultrasound image. For example, in response to user selection of the peripheral intravenous IV procedure 220, the user interface 260 displays a checklist 270 that includes the steps that must be performed to successfully complete such procedure. As illustrated in the checklist 270 of FIG. 7, these steps include 1. Locating the target vessel (Locate Vessel); 2. Preparing the site (Prep Site); 3. Load cartridge 90 onto injector arm 40 (Load Cartridge); 4. Cannulate the target vessel (Cannulate), and 5. Document the procedure (Document).

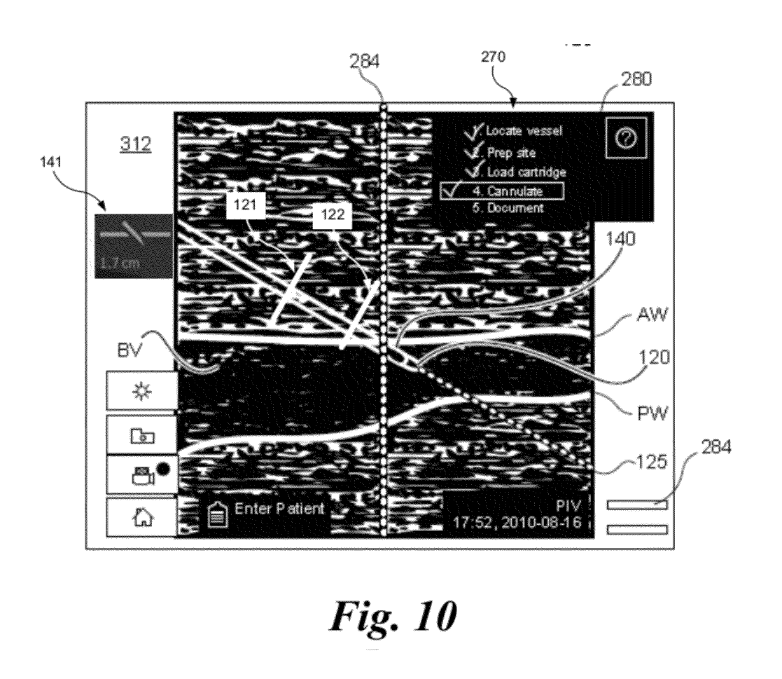

The interface 260 further includes a help button 280 that, when selected, is operable to invoke for display on the monitor 206 context-specific and/or selectable instructional images to assist the user in performing particular tasks, as discussed above, required for successful completion of a selected procedure. Referring to FIGS. 8 and 9, such instructional images may take the form of one or more still images or tutorial videos, and may occupy the entirety, or smaller portion, of the display screen of the monitor 206. For example, in response to user selection of the help button 280 during the “Locate Vessel” portion of the IV procedure 220, the interface 260 may display one or more images illustrating graphically and textually the correct approach to be taken by the user, as shown in FIG. 8. Subsequently, as illustrated in FIG. 9, the interface 260 may display one or more images illustrating the appearance of the ultrasound image if the user is employing the previously illustrated correct approach.

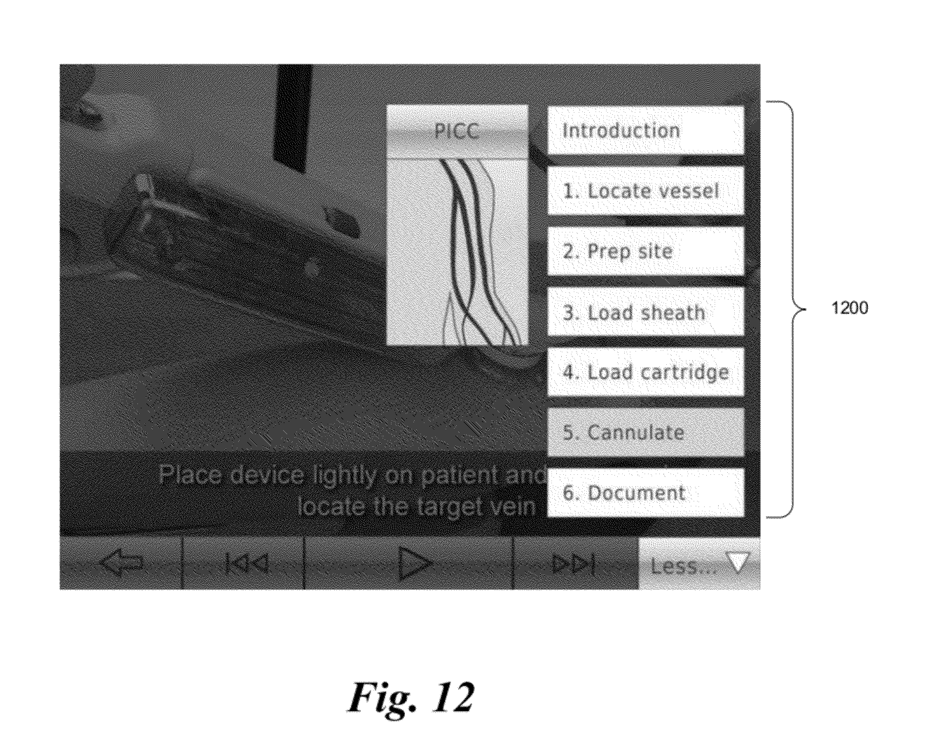

Alternatively, and as illustrated FIG. 12, selection of the help button 280 may invoke a menu 1200 of soft keys allowing the user to choose an image or tutorial video of a particular topic that may or may not be specific to the context of the procedure or procedure portion in which the user is currently engaged. Such topics may involve assisting the user in, for example, employing elements of the handset device 10 or interpretation of ultrasound images generated during a procedure.

Referring back to FIG. 7, in this interface 260 including an insonified vasculature image, a center located blood vessel BV is presented in short-axis cross section when the position of the rotatable transducer is indicated to be in short-axis mode by the presence of a short-axis icon 282, depicted in FIG. 7 as a thick circle. Applied to the ultrasound image of interface 260 is an overlay having positional information in the form of a vertical axis line 281. The vertical axis line 281 is shown bisecting the blood vessel BV and represents the approximate location of the center of the rotatable transducer of handset 10. Perpendicular to and intersecting the vertical axis line 281 are three horizontal lines 286, 290, 294 indicating various inclination angles of the injector arm 40 relative to the base 16 to achieve varying corresponding penetration depths (e.g., intersection of the needle with the ultrasound plane) for needle injection and cannulation. For example, horizontal axis line 286 represents a target depth of the needle/cannula when the injector arm 40 is at a 20-degree inclination angle (as illustrated at the top of FIG. 7), and horizontal axis line 290 represents a target depth of the needle/cannula when the injector arm 40 is at a 60-degree inclination angle (as illustrated at the bottom of FIG. 7). Between lines 286, 290 is horizontal axis line 294 that represents a target depth of the needle/cannula when the injector arm 40 is at a 33-degree inclination angle.

The intersection of any given horizontal axis line, seen in this example as horizontal axis lines 286, 290, or 294 with the vertical axis line 281 represents a targeting “cross-hair” or sighting-aid position where a cutting bevel end (not shown) of the needle 120 is expected to appear as the needle crosses the ultrasound plane when advancing at the corresponding angles described above. The horizontal axis can be adjusted to intersect at any given location of the vertical axis 281, indicative of the location of the base 16, by tilting or pivoting the injector arm 40 while holding the base firmly against the patient's skin. In this example, the intersection of horizontal line 294 with vertical line 281 is near the midline portion of the anterior wall (i.e., wall closest to the base 16) of the short-axis cross-sectional view of blood vessel BV. Generally, penetration of the blood vessel by the needle 120 near the midline of the anterior wall represents an ideal position for needle injection and cannulation procedures.

FIG. 10 illustrates a screenshot 312 of the interface 260 in long-axis cross-sectional view at the moment that the needle 120 with overlapping cannula 140 (or, alternatively, a representation of the needle and cannula generated by processor 202) is seen to be penetrating through the blood vessel BV at 30 degrees relative to the base 16 during the “cannulate” procedure of access menu 280. Processor 202 applies to the ultrasound image of screenshot 312 an overlay having positional information in the form of a trajectory line 125. Trajectory line 125 identifies the expected pathway that the needle 120 with overlapping cannula 140 will transit based on the angle between injector arm 40 and base 16, as well as needle/catheter advance position. Here, an image of the needle 120 with overlapping cannula 140 is shown penetrating the anterior watt AW of the blood vessel shown in long-axis mode as indicated by tong axis icon 284.

Still referring to FIG. 10, in an embodiment, if the user changes the angle between injector arm 40 and base 16 during a procedure, the processor 202 is configured to automatically adjust the depth setting of the displayed ultrasound image to keep the needle 120 with overlapping cannula 140 in full view within interface 260. For example, a steeper angle between injector arm 40 and base 16 is correlated to a deeper displayed image (i.e., more tissue surrounding the needle/cannula comes into view, white needle/cannula image is somewhat diminished in size), while a shallower angle is correlated to a shallower displayed image (i.e., less tissue surrounding the needle/cannula comes into view, while needle/cannula image is somewhat magnified in size). Optionally, this automatic depth-adjustment feature may be disabled by the user to maintain constant depth setting.

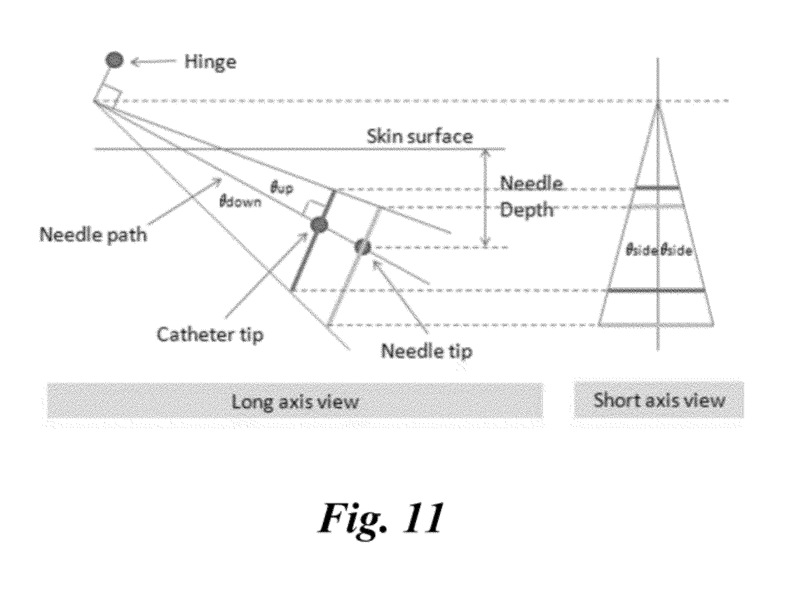

Processor 202 can also apply to the ultrasound image of screenshot 312 an overlay of a catheter-tip position indicator 121 and needle-tip position indicator 122. The position indicators 121, 122 indicate to the projected position of the cannula and needle tip in the ultrasound image. The position indicators 121, 122 may likewise be generated in the short-axis view depicted in FIG. 7. FIG. 11 illustrates the manner in which the position indicators 121, 122 are calculated according to an embodiment.

As illustrated in FIGS. 10 and 11, in long-axis view, the position indicator 122, in an embodiment: (1) intersects the calculated location of the needle tip; (2) is perpendicular to the trajectory line; (3) has an upper endpoint that forms an angle θᵤₚ between the hinge and the calculated needle tip location; and (4) has a lower endpoint that forms an angle θ_{down} between the hinge and the calculated needle tip location. Indicator 121 is developed analogously with respect to the catheter.

In the case of the short-axis view, tip positions are shown by horizontal lines positioned at the top and bottom of the lines calculated for the long-axis view. The width of the lines increases as depth increases, according to the angle θ_{side} shown in FIG. 11.

As further illustrated FIG. 10, the interface 260 may further include a tip-depth indicator 141. The tip-depth indicator 141 indicates whether the needle tip is above or below the skin surface. Above the skin surface, an icon of a first visual format (e.g., blue font) is shown, and the distance above the skin surface is shown as a negative number. Below the skin surface, an icon of a second visual format (e.g., orange font), different from the first format, is shown, and distance below the skin surface is shown as a positive number.

While the preferred embodiment of the invention has been illustrated and described, as noted above, many changes can be made without departing from the spirit and scope of the invention. For example, when ultrasound is displayed, it may always be scaled to fill as much of the interface 260 as possible—no frames or borders associated with, for example, soft keys or overlays, reduce the available area of ultrasound viewing. Moreover, soft keys and/or instructional images may or may not overlap a displayed ultrasound image. Additionally, during a procedure, needle/cannula trajectory may always be from upper-left to bottom-right. This area of the screen is kept clear of controls and indicators, which may be clustered in the upper-right and bottom-left portions of interface 260. Additionally, as the brightness of the ultrasound image changes, the processor 202 may automatically adjust the ultrasound gain to compensate for such changes. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. A computer-readable medium including executable instructions that, when executed by a processing device, enable the processing device to perform a method of generating to a display device a graphical user interface, the method comprising the steps of:
generating to a first region of the user interface an ultrasound image of a patient region of interest;
generating to the first region at least one overlay image configured to indicate a specific feature associated with the region of interest;
generating to a second region of the user interface a first set of selectable soft keys, the second region being exclusive of the first region; and
in response to user selection of a first-set soft key, generating to a third region of the user interface a corresponding functional image.

2. The medium of claim 1, wherein the third region is exclusive of the first region.

3. The medium of claim 1, wherein the ultrasound image, at least one overlay image and first set of soft keys are simultaneously generated to the user interface.

4. The medium of claim 1, wherein the method further comprises, prior to generating the ultrasound image to the user interface, generating to the user interface a second set of selectable soft keys, the second set of soft keys enabling a user to select an ultrasound procedure from a plurality of ultrasound procedures.

5. The medium of claim 1, wherein the at least one overlay image comprises an expected trajectory of an object intended for insertion into the region of interest.

6. The medium of claim 1, wherein the at least one overlay image comprises an image of an object being inserted into the region of interest.

7. The medium of claim 1, wherein the at least one functional image comprises a tutorial video describing to the user a method of performing a procedure using the ultrasound image.

8. The medium of claim 1, wherein the at least one overlay image comprises a checklist describing steps to be taken in performing a procedure using the ultrasound image.

9. The medium of claim 1, wherein the processing device is in electronic communication with an apparatus comprising an ultrasound-transducer base and an element configured to insert an object into the region of interest, the element being configured to be at an adjustable angle relative to the base, the method further comprising automatically adjusting a depth of the ultrasound image based on the angle of the element relative to the base.

10. The medium of claim 1, wherein the method further comprises automatically capturing, at a set of predetermined time intervals, a series of screenshots of the ultrasound image.

11. A system, comprising:
an ultrasound transducer; and
a processing device coupled to the transducer and configured to generate to a display device a graphical user interface, the processing device further configured to:
generate to a first region of the user interface an ultrasound image of a patient region of interest;
generate to the first region at least one overlay image configured to indicate a specific feature associated with the region of interest;
generate to a second region of the user interface a first set of selectable soft keys, the second region being exclusive of the first region; and
in response to user selection of a first-set soft key, generate to a third region of the user interface a corresponding functional image.

12. The system of claim 11, wherein the third region is exclusive of the firs region.

13. The system of claim 11, wherein the ultrasound image, at least one overlay image and first set of soft keys are simultaneously generated to the user interface.

14. The system of claim 11, wherein the processing device is further configured to, prior to generating the ultrasound image to the user interface, generate in the user interface a second set of selectable soft keys, the second set of soft keys enabling a user to select an ultrasound procedure from a plurality of ultrasound procedures.

15. The system of claim 11, wherein the at least one overlay image comprises an expected trajectory of an object intended for insertion into the region of interest.

16. The system of claim 11, wherein the at least one overlay image comprises an image of an object being inserted into the region of interest.

17. The system of claim 11, wherein the at least one functional image comprises a tutorial video describing to the user a method of performing a procedure using the ultrasound image.

18. The system of claim 11, wherein the at least one overlay image comprises a checklist describing steps to be taken in performing a procedure using the ultrasound image.

19. The system of claim 11, wherein the ultrasound transducer comprises a base, the system further comprising an element configured to insert an object into the region of interest, the element being configured to be at an adjustable angle relative to the base, the processing device further configured to automatically adjust a depth of the ultrasound image based on the angle of the element relative to the base.

20. The system of claim 11, wherein the processing device is further configured to automatically capture, at a set of predetermined time intervals, a series of screenshots of the ultrasound image.
